(19) United States
(12) Patent Application Publication
(10) Pub. No.: US 2013/0230503 A1
(43) Pub. Date: Sep. 5, 2013

(54) METHOD AND PREPARATION FOR THE TREATMENT OR PREVENTION OF ANXIETY OR NEUROGENESIS

(71) Applicants: Judith Homberg, Nijmegen (NL); Amanda Johanne Kiliaan, Nijmegen (NL); Ladislaus Maria Broersen, Utrecht (NL)

(72) Inventors: Judith Homberg, Nijmegen (NL); Amanda Johanne Kiliaan, Nijmegen (NL); Ladislaus Maria Broersen, Utrecht (NL)

(73) Assignee: N.V. Nutricia

(21) Appl. No.: 13/820,463

(22) PCT Filed: Sep. 3, 2010
(86) PCT No.: PCT/NL2010/050558

§ 371 Date: May 13, 2013

(51) Int. Cl. A61K 31/661; A23L 1/29; A61K 31/202; A61K 31/4415; A61K 31/519

(52) U.S. Cl. CPC A61K 31/661; A61K 31/4415; A61K 31/519; A61K 31/202; A23L 1/296
USPC 424/94.1; 514/349

(57) ABSTRACT

The present invention relates to the use of a preparation, especially a nutritional preparation, for the prevention or treatment of anxiety in a subject, especially in a depressed subject, and depression. More specifically, the present invention relates to the use of a preparation, especially a nutritional preparation, for the prevention or treatment of anxiety or depression in a subject that is non-responsive to SSRI medication. Furthermore, the invention relates to the use of a preparation, especially anutritional preparation, for regulating neurogenesis. The preparation comprises the following components: a) at least one ω-3 polyunsaturated fatty acid (PUFA); b) at least two phospholipids, selected from the group consisting of phosphatidylserine, phosphatidylinositol, phosphatidylcholine and phosphatidylethanolamine or any mixture thereof; and c) one or more compounds which are a factor in the serotonin metabolism, selected from the group of B vitamins and tryptophan.

### BACKGROUND OF THE INVENTION

The present invention relates to the use of a preparation, especially a nutritional preparation, for the prevention or treatment of anxiety in a subject, especially in a depressed subject, and depression. More specifically, the present invention relates to the use of a preparation, especially a nutritional preparation, for the prevention or treatment of anxiety or depression in a subject that is non-responsive to SSRI medication. Furthermore, the invention relates to the use of a preparation, especially a nutritional preparation, for regulating neurogenesis.

Anxiety and depression have both been associated with disturbances of the serotonergic system [1-3]. The serotonergic system is highly complex, consisting of 14 receptor subtypes, but there is a single transporter responsible for the reuptake of 5-HT; the 5-HT transporter (5-HTT) [4]. Thereby, changes in 5-HTT expression and/or function have profound consequences for the availability of 5-HT in the extracellular space, mood and emotional control. One example is the low activity (short; s) variant of the serotonin transporter length polymorphic region (5-HTTLPR) in humans, which is well known for its association with anxiety-related personality traits [5], and increased risk for depression in the context of early life stress [6, 7]. These behavioural manifestations correlate with amygdala hyperreactivity, amygdala and hippocampus hyperperfusion, and alterations in volume [8, 9]. It is possible that a ‘gain in function’ in these limbic nodes results in decreased stress-resilience [10]. 5-HTTLPR genotype also influences the decrease in hippocampal volume that is typically seen in prolonged major depressive episodes, although some ambiguity remains with regard to the precise nature of this effect [9, 11, 12].

Meta-analyses have shown that depression-prone 5-HTTLPR short (s)-allele carriers respond relatively poor to SSRIs, the drugs of choice when treating depression [13]. SSRIs may exert their antidepressant effects through changes in adult hippocampal neurogenesis [14], which are believed to be mediated by the altered 5-HT_{1A} signalling caused by chronic SSRI treatment [15]. Newly formed neurons are known to integrate into existing neural pathways and contribute to hippocampal learning and emotional control [16, 17]. The association between the s-allele and neurogenesis remains to be assessed, but the ‘gain of function’ concept suggests that the mechanisms of action of SSRIs work out differently in s-allele carriers.

In the present application, we specifically demonstrate the possibility to treat depressive symptoms in a subject that is non-responsive to SSRI medication with the use of a nutritional intervention. Interestingly, diet is not only an important lifestyle factor implicated in the aetiology and treatment of depressive and associated disorders, but it has also been shown that different nutrients may affect several of the mechanisms involved in the pathogenesis of depression and of those underlying the actions of antidepressants like SSRIs. For instance, omega-3 polyunsaturated fatty acid (ω-3-PUFA) deficiency may have a negative effect on serotonergic neurotransmission [18], while supplementation of nutrients that change neuronal membrane phospholipid structure and fatty acid composition may affect membrane characteristics [19], which may facilitate neuronal signalling by increasing ion channel availability [20], or improve 5-HT signal transduction by increasing 5-HT_{1A} receptor density [21] and binding affinity [22]. Similarly, some nutrients are known precursors and cofactors necessary for the synthesis of neurotransmitters and neuronal membranes. Additionally, nutrients like ω-3-PUFAs have been associated with changes in hippocampal neurogenesis in rats and mice [23-27], although the precise mechanisms that drive such neurogenesis are still a subject of speculation.

Based on epidemiological evidence showing that dietary intake of nutrients like ω-3-PUFAs and B-vitamins is associated with the risk for depressive disorders [28-30], it has been suggested that supplementation of these nutrients might help to reduce associated symptoms. To date, however, supplementation studies with single nutrients have not shown clear benefits in terms of decreasing disease risk or aiding in symptom management. Nevertheless, nutritional components may have therapeutic potential, as evidenced by the observation that ω-3-PUFA intake in conjunction with antidepressant medication improved the outcome of recurrent unipolar depressive disorder patients [31]. Additionally, it has been shown that combined administration of specific nutrients enhanced their efficacy for reducing depressive-like symptoms [32], suggesting potential synergistic effects.

Since several nutrients may help to compensate some of the deficits reported for s-allele carriers and since combined administration of such nutrients may be more effective than single nutrient intervention for reducing depressive-like symptoms [32], we hypothesized that especially a diet combining the administration of ω-3-PUFAs with phospholipids and B-vitamins might have beneficial effects in 5-HTTLPR-s allele carriers. To test this hypothesis, we used female serotonin transporter knockout (5-HTT^{-/-}) rats and their wild-type controls (5-HTT^{+/+}). Like 5-HTT^{-/-} mice and human s-allele carriers, these animals show increased anxiety and depression-like symptoms (for review see Kalueff et al [33]). The higher prevalence of depression among women compared to men [34] was the rationale for choosing female rats in the present study. After being fed either a mixed PUFA diet or a control diet, the animals were subjected to a series of behavioural tests measuring either anxiety (elevated plus maze, fear conditioning) or depression-like symptoms (social interaction test, forced swim test). The neurobiological correlates of genotype and diet effects were addressed using immunohistochemical staining for the neurogenesis marker doublecortin (DCX) [35]. In addition we measured hippocampal volume, which may be affected in severe depression.

As used herein, “or” is meant to include also “and”. Hence, the expression “A or B” encompasses 3 options: A, B and “A and B”, unless A and B are mutually exclusive.

### PRIOR ART

EP 1 275 399 B1 and EP 1 275 399 B1 (both of Nutricia NV) disclose the use of a preparation according to the invention for use in the prevention and treatment of vascular disorders and certain conditions associated therewith, such as bipolar or unipolar depression and for the treatment of depressions, related to anxiety. As is disclosed, the composition is effective because it provides activity on the function of the tunica intima and endothelial cells in general, which is important for influencing the aetiology and development of a wide range of vascular disorders and several other secondary disorders, in particular depression.

### DESCRIPTION OF THE INVENTION

The present inventors have now found a preparation for the treatment of depression and anxiety, in particular not associated with vascular disorders, that is effective because of its effect on the serotonergic system, in particular the 5-HTT and 5-HTTLPR component of the serotonergic system.

Thus, the present invention provides a preparation suitable for the prevention or treatment of depression or anxiety in a subject, comprising the following components:

a) at least one ω-3 polyunsaturated fatty acid (PUFA);
b) at least two phospholipids, selected from the group consisting of phosphatidylserine, phosphatidylinositol, phosphatidylcholine and phosphatidylethanolamine or any mixture thereof; and
c) one or more compounds which are a factor in the serotonin metabolism, selected from the group of B vitamins and tryptophan.

Preferably, said subject is a mammal, more preferably a human.

### DETAILED DESCRIPTION OF THE INVENTION

The combined administration of these components results in the prevention and treatment of anxiety or depression, in particular on the level of the serotonergic system.

### Component a)

Component a) comprises at least one ω-3 polyunsaturated fatty acid (PUFA). The fatty acid can be a free fatty acid, but is preferably bound to a suitable backbone, for instance a triglyceride. It can also be in the form of phospholipids, as will be described later.

If a mixture of ω-3 and ω-6 polyunsaturated fatty acids (PUFA's) is used, it was found that such mixture should be included in a ratio of ω-3 fatty acids to ω-6 fatty acids of about 2.5 to 5.5 wt/wt.

Preferred ω-3 PUFA's are eicosapentaenoic acid (EPA) and docosahexaenoic acid (DHA). Best results are obtained when DHA and EPA are included in about equimolar amounts, for example a ratio of DHA to EPA of 0.5 to 2 wt/wt. Preferred ω-6 PUFA's are dihomogammalinolenic acid (DHGLA) and arachidonic acid (AA). These should be included in an amount of about one fourth of the amount of EPA and DHA, for example a ratio of [DHA+EPA] to [DHGLA+AA] of 2.5 to 5.5, preferably 3.3-4.7 wt/wt. The daily dosage of the total of EPA+DHA+DHGLA+AA is preferably at least 120 mg, more preferably at least 350 mg. Per daily dose the preparation in particular contains 20 to 2000 mg, preferably 50 to 1000 mg EPA, 50 to 2000 mg, preferably 200 to 1000 mg DHA and 50 to 2000 mg, preferably 100 to 1000 mg DHGLA. Further PUFA's that can be present are linolenic and α-linoleic acid. However, the ratio of the total amount of EPA+DHA+DHGLA+AA to the total amount of linolenic and a-linoleic acid should be larger than 0.1 wt/wt, preferably larger than 0.2, most preferably larger than 0.4.

### Component b)

Component b) comprises at least two phospholipids selected from the group consisting of phosphatidylserine, phosphatidylinositol, phosphatidylcholine and phosphatidylethanolamine.

Preferably, said phospholipids are phosphatidylcholine and phosphatidyl-ethanolamine. Preferably, component b) comprises at least three different phospholipids selected from the group consisting of phosphatidylserine, phosphatidylinositol, phosphatidylcholine and phosphatidylethanolamine. More preferably, component b) comprises phosphatidylcholine, phosphatidylethanolamine and phosphatidylserine. The function of this component may be seen as providing a direct source of neuronal cell phospholipids (building blocks) for the synthesis of neurons (neurogenesis). It is highly preferred to include a mixture of phospholipids, especially with regard to the choline/ethanolamine moiety couple on the one hand, and the serine/inositol moiety couple on the other hand. For best results, the ratio of (phosphatidylcholine or phosphatidylethanolamine) to (phosphatidylserine or phosphatidylinositol) is 0.5-20 (wt/wt). Per daily dose at least 0.2 g and preferably more than 1 g phospholipids should be administered, for example 4 g. When the product is meant to be used by patients suffering from anxiety and depression symptoms, the amount of phosphatidylserine per daily dose product should be at least 0.1 g for best results and preferably more than 0.5 g. Another preferred characteristic of the preferred phospholipids is the PUFA moiety. It is preferred to use phospholipids which provide the PUFA's as described above for component a). For example, they can be prepared by applying interesterification methods known in the art using for example raw phospholipid mixtures and ingredients that are rich in the particular PUFA's. Use of these specific phospholipids ensures a high activity next to a relatively stable product. In preparations for oral use, it is not required to use higher organised lipid components such as sphingomyelines due to the high metabolic rates of this type of compound in the gut, gut epithelial cells and liver. Also, other lipids, that are essentially free from DHA, EPA, DHGLA or AA, such as neutral triglycerides are preferably not included in the phospholipid component or in relatively low amounts, e.g. less than 40% and in particular less than 5% of the lipid component. Phospholipids can originate from egg yolk or soy, such as soy lecithine, and can be isolated by applying methods that are known in the art, for example acetone extraction and optionally applying subsequent chromatographic techniques or adsorption methods. The phospholipid component can also consist, where required, of mixtures of synthetic phospholipids and (extracts of) phospholipids of natural origin.

### Component c)

Component c) comprises at least one compound, which is a factor in the serotonin metabolism.

In one embodiment, component c) comprises at least one ore more B vitamins selected from the group of pyridoxine (B6), folic acid/folate (B11), thiamine (B1), riboflavine (B2), nicotinic acid (B3), pantothenic acid (B5), cobalamine (B12) and Vitamin H.

Preferably, component c) comprises at least pyridoxine (vitamin B6). Vitamin B6 aids in the manufacture of serotonin. A deficiency of this B vitamin reduces serotonin production and affects mood and cravings. Vitamin B6 should be included in an amount of more than 2 mg, in particular more than 2.5 mg per daily dose.

Preferably, component c) comprises at least folic acid (vitamin B11), in particular in an amount of at least 200 μg and most preferably more than 400 μg per daily dose. Folic acid is also meant to include physiological equivalents thereof, such as pharmaceutically acceptable salts thereof, 5-methyltetrahydrofolate and polyglutamate forms thereof as they occur naturally. It is most preferred that at least vitamin B6 and folic acid are included, while the largest part of the population will benefit if these components are simultaneously included.

In one embodiment, component c) comprises tryptophan. Tryptophan raises blood levels, then brain levels of tryptophan, which increases serotonin production.

### Compound d)

Besides the components a) to c) described above, the preparation according to the invention may optionally contain a further component d) which is beneficial for the prevention or treatment of anxiety, especially in depressed persons, and depression, and for stimulating neurogenesis. Furthermore, in view of the disclosure of EP 1 275 399 B1 and EP 1 275 399 B1, a combined effect on the serotonergic system and on the function of the tunica intima and endothelial cells in general could be obtained.

Component d) may comprise a compound, which is a factor in the methionine metabolism. Total methionine metabolism (TMM) has been described in EP 0 891 719. It is known that a proper functioning of TMM is mandatory for the endogenous biosynthesis of many crucial compounds such as S-adenosyl methionine (for creatine, carnitine, etc) and glutathion. Component d) may comprise at least one compound selected from the group consisting of folic acid, vitamin B12, vitamin B6, magnesium and zinc. It is even more advantageous when this component contains all of the members of the above mentioned group. The component can further contain SAMe (S-adenosyl methionine), choline, betaine or copper. If component d) comprises zinc and copper, the weight ratio of zinc to copper is between 5 to 12. Choline or betaine can be included.

Component d) may comprise citrate. Citrate is also meant to include citric acid. The products according to the invention should have a pH between 3.0 and 7.5 and preferably between 5 and 7. Citrate should be administered in an amount of 0.5 to 30 g, preferably 1.5 to 10 g per daily dose, for example more than 2.4 g. Biochemistry literature discloses that citric acid, as well as some other compounds, provides reducing equivalents to the cytosol and participates in the “Krebs cycle”, thus yielding NADH and energy in the mitochondriae. It is also known for a long time that citric acid helps regulate glycolyses by feedback inhibition of the phosphofructokinase reaction. For a proper functioning of vascular endothelial cells it is important to have at the same time sufficient amounts of ATP and reducing equivalents in the form of NADPH available in the cytosol of these cells and that citrate can ensure this to occur, and more effectively than a functional analogue like a Krebs cycle intermediate like oxaloacetate, malic acid or fumarate.

Component d) may comprise huperzine A or a functional analogues thereof, especially in those products that are designed to be used for the prevention and treatment of dementia syndromes, especially in those phases of the disease where acetylcholine metabolism is severely impaired. Huperzine A should be included in amounts of 0.04 to 2, preferably 0.07 to 1, most preferably 0.08 to 0.5 mg per daily dose. As an analogue also an extract of certain herbs such as Huperzia serrata can be used, when standardised on huperzine A content and purity. An amount of 0.04 to 20 mg, preferably 0.07 to 2 mg per daily dose of such an extract can be used. Also lipophilic derivatives of huperzine A can be used, e.g. those obtained by modification of the primary and/or secondary amino groups.

Component d) may comprise a one or more of carnitine, vitamin B1, vitamin B5 and coenzyme Q10 or functional analogues thereof. In one embodiment, component d) may comprise a one or more of carnitine and coenzyme Q10 or functional analogues thereof. As functional equivalents of carnitine can be mentioned pharmaceutically acceptable salts thereof or alkanoyl and acyl carnitines [acetyl-L-carnitine], which are particularly useful, or mixtures thereof. Carnitine is advantageously included in products that are meant to be used for patients suffering from dementia syndromes. In these products, preferably a lipophilic derivative is used as carnitine source. It is most preferred to use acetyl-L-carnitine. This component provides acetyl groups in the brain for biosynthetic purposes. Carnitine should be included in an amount of 0.1 to 3 g, preferably 0.2 to 1 g per daily dose. Vitamin B5 can be included for instance as calcium pantothenate or other stable form. Preferred dosages are 8 to 80 mg, preferably 12 to 40 mg per daily dose product.

Component d) may comprise a lipophylic thiamine source such as benfothiamine, allithiamine, fursulthiamine or octothiamine, especially for preparations that are meant to be used for the treatment or prevention of further progression of dementia syndromes. A degeneration of cerebral function in human subjects, as is observed during Parkinson's and Huntington's disease, can be retarded by the preparations according to the invention. In preparations for these types of subjects, it is advantageous to include also respectively taurine and gamma-amino butyric acid or derivatives thereof such as piracetam. If coenzym Q10 is included, the amount can be 0.8 to 200 mg and preferably 5 to 70 mg. The amounts can be that low because of the beneficial effect of the phospholipids (component b) on the membrane function.

Component d) may comprise a compound that provides anti-oxidant properties. Such compound may be selected from the group of vitamin C, vitamin E, lipoic acid, selenium salts and carotenoids.

Component d) may comprise an extract of gingko biloba. This extract is obtained from the leaves and is enriched in flavonoids and especially terpenoids, in particular ginkgolides. It appears for example that an extract that comprises at least 4% ginkgolides is effective.

Preferably, the preparation according to the invention comprises the above components in an amount above the recommended daily intake. Per daily dose, the preparation according to the invention preferably comprises:
at least 120 mg long chain polyunsaturated fatty acids;
at least 200 mg phospholipids;
at least 200 μg folic acid; and
at least 0.5 g citrate.

More preferably, the preparation according to the invention comprises per daily dose:
at least 20 mg, preferably at least 50 mg of eicosapentaenoic acid (EPA)
at least 50 mg, preferably at least 200 mg of docosahexaenoic acid (DHA)
at least 50, mg preferably at least 100 mg of arachidonic acid (AA)
at least 200 mg, preferably at least 1000 mg of phosphatidylserine (PS),
at least 200 μg, preferably at least 400 μg of folic acid
at least 100 mg, preferably at least 200 mg of magnesium
at least 5 mg, preferably at least 10 mg of zinc
at least 2 mg, preferably at least 2.5 mg of vitamin B6
at least 2 μg, preferably at least 4 μg of vitamin B12
at least 1.0 g, preferably at least 1.5 g of citrate.

The preparation according to the invention can be a pharmaceutical, as well as a nutritional preparation.

In particular in case of a nutritional composition, which may be a food or beverage, the amount of preparation according to the invention contained therein is suitably present in the composition in a quantity to provide the daily dosage in a single serving.

The term “serving” as used herein denotes an amount of food or beverage normally ingested by a human adult with a meal at a time and may range, e.g., from about 1 g (such as a nutritional shot) to about 500 g.

In one aspect of the present invention, the preparation according to the invention (i.e. comprising components a), b), c) and optionally d)) may be used in a pharmaceutical composition comprising one or more pharmaceutically acceptable carrier materials.

The pharmaceutical composition, preferably for enteral application, may be solid or liquid galenical formulation. Examples of solid galenical formulations are tablets, capsules (e.g. hard or soft shell gelatine capsules), pills, sachets, powders, granules and the like which contain the active ingredient together with conventional galenical carriers. Any conventional carrier material can be utilized. The carrier material can be organic or inorganic inert carrier material suitable for oral administration. Suitable carriers include water, gelatine, gum Arabic, lactose, starch, magnesium stearate, talc, vegetable oils, and the like. Additionally, additives such as flavouring agents, preservatives, stabilizers, emulsifying agents, buffers and the like may be added in accordance with accepted practices of pharmaceutical compounding. While the individual active ingredients are suitably administered in a single composition they may also be administered in individual dosage units.

If the composition is a pharmaceutical formulation, such formulation may contain the daily dosage in one or more dosage units. The dosage unit may be in a liquid form or in a solid form, wherein in the latter case the daily dosage may be provided by one or more solid dosage units, e.g. in one or more capsules or tablets.

In another aspect of the present invention, the preparation according to the invention (i.e. comprising components a), b), c) and optionally d)) may be formulated as a nutritional composition comprising at least one component selected from the group of fats, proteins, and carbohydrates. It is understood that a nutritional composition differs from a pharmaceutical composition by the presence of nutrients which provide nutrition to the subject to which the composition is administered, in particular the presence of protein, fat, digestible carbohydrates and dietary fibres. It may further contain ingredients such as minerals, vitamins, organic acids, and flavouring agents. Although the term “nutraceutical composition” is often used in literature, it denotes a nutritional composition with a pharmaceutical component or pharmaceutical purpose. Hence, the nutritional composition according to the invention may also be denoted a neutraceutical composition.

Advantageously, the nutritional composition according to the invention may comprise protein, preferably intact protein. Proteins enable the manufacturing of palatable products. Especially elderly benefit from the protein as it strengthens their motor skills. Preferably, the nutritional composition according to the invention comprises milk protein. Preferably, the nutritional composition according to the invention comprises a protein selected from the group consisting of whey protein, casein or caseinate. Preferably, the nutritional composition according to the invention comprises caseinate, more preferably the nutritional composition according to the invention comprises at least 70 weight %, more preferably at least 90 weight % casein and/or caseinate, based on total protein.

Preferably, the proteins are included in intact (unhydrolyzed) form, in order to have a palatable product. Such high molecular weight proteins increase the viscosity of the heat-treated liquid product, compared to the hydrolyzed forms. The present inventors were able to make an acceptable product, with good palatability and limited viscosity, by applying the measures according the invention, still avoiding precipitation.

Preferably, the nutritional composition according to the invention comprises between 0.2 and 16 gram protein per 100 ml, preferably between 0.2 and 10 gram protein per 100 ml, more preferably between 1 and 6 grams protein per 100 ml, more preferably between 2 and 5 grams protein per 100 ml.

Advantageously, the nutritional composition according to the invention may comprise fat, comprising fat constituents other than mentioned under component a). With regard to the type of fat, a wide choice is possible, as long as the fat is of food quality. The fat may be a solid, a semi-solid or a liquid (oil) at room temperature (25° C.).

The fat may include one or more medium chain triglycerides (MCT), one or more long chain triglycerides or any combination of the two types. The MCT or MCT's may in particular be selected from MCT's having a triglyceride chain that is 6, 7, 8, 9 or 10 carbon atoms long. The LCT or LCT's typically are at least 12 carbon atoms long.

MCTs are beneficial because they are easily absorbed and metabolized. Moreover, the use of MCTs will reduce the risk of nutrient malabsorption.

LCT sources, such as rapeseed oil, more in particular rapeseed oil low in erucic acid, sunflower oil, corn oil, palm kernel fat, coconut fat, palm oil, or mixtures thereof are preferred because they provide more energy per unit of fat.

In a specific embodiment, the fat comprises 30 to 60 weight % of animal or algal fat, 40 to 70 weight % of vegetable fat and optionally 0 to 20 weight % of MCTs based on total fat of the nutritional composition according to the invention. The animal fat preferably comprises none or a low amount of milk fat, i.e. lower than 6 weight %, especially lower than 3 weight %. In particular, a mixture comprising one or more oils selected from the group of corn oil, egg oil, canola oil and marine oil may be present. Egg oils, fish oils and algal oils are a preferred source of non-vegetable fats. Marine oils are source of DHA and/or EPA which are present in the nutritional composition according to the invention. For a desirable taste, the concentration preferably is 25 weight % or less, more preferably 15 weight % or less of the fat.

Advantageously, the nutritional composition according to the invention comprises one or more digestible carbohydrates. The digestible carbohydrates positively influence the operational skills of a subject, and add to the advantageous effect of the nutritional composition according to the invention. The total amount of digestible carbohydrates is preferably between 25 and 80 weight % on dry matter basis, preferably 40 to 80 weight %. In case of a liquid nutritional composition according to the invention, the composition preferably contains between 1 and 50 gram digestible carbohydrates per 100 ml of a liquid product, more preferably between 5 and 30 grams per 100 ml, more preferably 10 to 30 grams of digestible carbohydrates per 100 ml.

Examples of digestible carbohydrates are digestible pentoses, digestible hexoses and digestible oligosaccharides, e.g. digestible disaccharides and digestible trisaccharides. More specifically one or more digestible carbohydrates may be chosen selected from the group of galactose, mannose, ribose sucrose, trehalose, palatinose, lactose, maltodextrose, maltose and glucose.

Optionally, a nutritional composition according to the invention comprises one or more non-digestible carbohydrates (dietary fibres) such as oligosaccharides. As used herein, the term oligosaccharides in particular refers to saccharides comprising 3 to 25 monosaccharide units per molecule. The oligosaccharide(s) may in particular be selected from the group of fructo-oligosaccharides (FOS), galacto-oligosaccharides (GOS), trans-galacto-oligosaccharides (TOS), xylo-oligosaccharides (XOS), soy oligosaccharides, and the like. Optionally, also higher molecular weight compounds such as inulin, resistant starch and the like may be incorporated in the composition according to the invention. In a further embodiment of the present invention, the composition according to the invention comprises a mixture of neutral and acid oligosaccharides, such as disclosed in WO 2005/039597 (N.V. Nutricia); compositions disclosed therein are incorporated herein by reference.

Furthermore, one or more of the following components may in particular be present: taurine, cystein, manganese, molybdenum, zinc, selenium, magnesium, chromium, iron, copper, vitamin A, vitamin B1, vitamin B2, vitamin B3, vitamin B5, vitamin B6, folic acid, vitamin B12, vitamin C, vitamin D, vitamin E and biotin.

The nutritional composition for use in a accordance with the invention may in particular be selected from the group of spreads; yoghurts, liquids, powders, custards, ice-creams, butter, and other dairy products; dairy-substitute products; fruit drinks; bars, sweeties, concentrate, pate, sauce, gel, emulsion, and cookies.

Preferably, the nutritional or neutraceutical composition according to the invention is a liquid, preferable a dairy-based liquid nutritional composition for medical purposes.

Preferably, a liquid nutritional composition according to the invention has an energy density of 80 to 450 kcal per 100 ml of the composition, more preferably between 90 and 250 kcal per ml of the liquid nutritional composition. This is in particular considered advantageous because persons suffering from neuropathies or neurological problems often experience problems with eating. Their sensory capabilities and/or control of muscles generally have become imparted, as well as in some instances their ambition to apply proper eating habits. Part of these patients may experience a general loss in appetite and a relatively large part of this patient group became malnourished. A liquid nutritional composition is relatively easy to administer, and by having an energetic value in the specified range, such people can relatively easily obtain sufficient caloric intake.

Liquid nutritional compositions preferably have a long shelf life. However, increasing shelf life by heat treatments often results in destabilisation of the products and/or palatability, leading to a product which is undesirable. A nutritional composition according to the invention can be subjected to a heat treatment without major adverse effects on the palatability. Hence, the nutritional composition according to the invention is preferably heat-treated, more preferably the composition is subjected to a sterilization treatment. In a preferred embodiment, the nutritional composition according to the invention is subjected to an ultra-high temperature treatment (UHT-treatment). Such UHT-treatment is preferably applied in line, i.e. before the liquid final product is filled in the package of the unit.

### Medical Use

The preparations according to the invention can be used for the prevention or treatment of anxiety in a subject, especially in a depressed subject and depression, especially in a subject that is non-responsive to SSRI medication, and especially in a subject that has not been diagnosed with a vascular disorder, such as atherosclerosis, arteriosclerosis, atherosclerosis obliterans, angina pectoris, myocard infarct, cerebral vascular accidents, thrombosis, M. Bürger, varices, thrombo-phlebitis, syndrome of Raynaud, hypercholesterolaemia, hyperlipidaemia, elevated blood pressure, temporary disorders associated with ischaemia, vene thrombose, postpartum thrombose, varicose veins (varices), and thrombo-angiitis obliterans. When referring to prevention and treatment of anxiety or depression, it is understood that either one of the underlying cause (serotonergic system) or the symptoms of said conditions, or both are prevented and treated, in particular the symptoms being diminished, lowered, decreased or removed.

### DESCRIPTION OF FIGURES

FIG. 1: Effects of dietary interventions in 5-HTT^{-/-} and 5-HTT^{+/+} rats on elevated plus maze performance. The mixed PUFA diet reduced the increased levels of anxiety observed in 5-HTT^{-/-} rats. * p<0.05 5-HTT^{-/-} versus 5-HTT^{+/+} rats on control diet; # p<0.05 5-HTT^{-/-} rats on control diet versus mixed PUFA diet.

FIG. 2: Effects of dietary interventions in 5-HTT^{-/-} and 5-HTT^{+/+} rats on mobility (left) and immobility (right) measures in the forced swim test of depression. The mixed PUFA diet reduced the depressive-like behaviour (decreased mobility/increased immobility) observed in 5-HTT^{-/-} rats. * p<0.05 5-HTT^{-/-} versus 5-HTT^{+/+} rats on control diet; # p<0.05 5-HTT^{-/-} rats on control diet versus mixed PUFA diet.

FIG. 3: Effects of dietary interventions in 5-HTT^{-/-} and 5-HTT^{+/+} rats in the social interaction test. The mixed PUFA diet improved social behaviour in 5-HTT^{-/-} rats, as evidenced by an increased contact time (top left) and a normalization of self-grooming (bottom left). * p<0.05 5-HTT^{-/-} versus 5-HTT^{+/+} rats on control diet; # p<0.05 5-HTT^{-/-} rats on control diet versus mixed PUFA diet.

FIG. 4: Effects of dietary interventions in 5-HTT^{-/-} and 5-HTT^{+/+} rats on the extinction of conditioned fear. The mixed PUFA diet improved the slow rate of fear extinction observed in 5-HTT^{-/-} rats. * p<0.05 5-HTT^{-/-} versus 5-HTT^{+/+} rats on control diet; # p<0.05 5-HTT^{-/-} rats on control diet versus mixed PUFA diet; ^{a} p<0.05 5-HTT^{+/+} rats on control diet versus mixed PUFA diet.

FIG. 5: Effects of dietary interventions in 5-HTT^{-/-} and 5-HTT^{+/+} rats on hippocampal neurogenesis expressed as nascent neuronal cell bodies identified by DCX staining. The mixed PUFA diet normalized the deviant neurogenesis observed in 5-HTT^{-/-} rats. * p<0.05 for the indicated comparisons.

FIG. 6: Effects of dietary interventions in 5-HTT^{-/-} and 5-HTT^{+/+} rats on estimated hippocampal volume. The mixed PUFA diet increased hippocampal volume in 5-HTT^{+/+} rats. * p<0.05 for the indicated comparisons.

### EXPERIMENTAL

### Materials and Methods

### Animals

Serotonin transporter knockout rats (Slc6a4^{1Hubr}) were generated by ENU-induced mutagenesis [36]. Experimental animals were derived from crossing heterozygous 5-HT transporter knockout (5-HTT^{+/-}) rats that were outcrossed with commercial (Harlan, Ter Horst, The Netherlands) wild-type Wistar rats for at least eight generations. All animals were housed two per cage in a temperature (21±1° C.) and humidity-controlled room (45-60% relative humidity), and had ad libitum access to water and food throughout the experiment. A 12/12 h light-dark cycle was maintained, with lights on from 08:00 a.m. to 20:00 p.m. All experiments were approved by the Committee for Animal Experiments of the Radboud University Nijmegen Medical Centre, Nijmegen, The Netherlands, and all efforts were made to minimize animal suffering and to reduce the number of animals used.

### Diets

From the age of 65 days, the animals were fed for 3 months with either a control diet or a mixed PUFA diet (Research Diet Services, Wijk bij Duurstede, The Netherlands). Both diets were AIN-93M based [37], isocaloric and identical with respect to their protein, carbohydrate, fibre, and mineral content. Differences between the diets are summarized in Table 1. In contrast to the control diet, the mixed PUFA diet provided the combination of ω-3-PUFAs (fish oil), phospholipids (soy lecithin) and increased levels of B-vitamins (pyridoxine (vitamin B6) and folic acid (vitamin B11).

TABLE 1
Overview of the differences between the two isocaloric diets that
were used in the current study. In contrast to the control diet, the
mixed PUFA diet provided the combination of ω-3-PUFAs
(fish oil), phospholipids (soy lecithin) and increased levels of
B-vitamins (pyridoxine and folic acid).

| | Control | mixed PUFA |
|---|---|---|
| 5% Fat: | | |
| Soy oil | 1.90 | |
| Coconut oil | 0.90 | 0.10 |
| Corn oil | 2.20 | 1.87 |
| Fish oil | | 3.03 |
| Extra's: | | |
| Soy lecithin | | 0.755 |
| Pyridoxine | | 0.00328 |
| Folic acid | | 0.00067 |

### Behaviour

Elevated Plus Maze

The maze was custom made from polyvinylchloride. It was elevated to a height of 50 cm with two open (50×10) and two enclosed arms (50×10×40) arranged such that the arms of the same type were opposite to each other. The illumination intensity measured in the open arms was 80 lux. Female rats were tested as described earlier [38]. Rats were placed in the centre of the maze, facing one of the open arms, for a free exploration period of 5 min. The movements and position of the animals were recorded and registered automatically using Ethovision© 3.1 software (Noldus Information Technology BV, Wageningen, The Netherlands). Results were expressed as the mean of time spent in open arms.

Forced Swim Test

Cylindrical glass tanks (50 cm tall×18 cm diameter), filled to a depth of 30 cm with 22 (+/−1)° C. water, were used. Female rats were tested as described earlier [38]. In short, after a 15-min water experience on day 1, the animals were tested 24 hrs later in the water cylinders for 5 min. The movements of the rats were videotaped for off-line measurement of the duration of immobility (s). The behavioural variable ‘immobility’ was defined as follows: making no movements for at least 2 seconds or making only those movements that were necessary to keep the nose above the water. Active climbing, diving and swimming along the wall were scored as mobility (s).

Social Interaction Test

Social interaction was measured in a test cage (50×50×75 cm ((l×w×h)) which had acrylic plastic walls and was filled with sawdust (2 cm). The experimental room was illuminated by a 25-W fluorescent red light, mounted 60 cm above the test cage. 24 hours before the test, the female rats were habituated to the test cage during 10 min. Social interaction pairs were designed such that both rats were genotype and diet matched; rats from the same litter or home cage were not paired. On the test day, test pairs were isolated for 2 hrs prior to the test to increase in the amount of social behaviour, and subsequently tested for 15 min. Behaviour of the animals was recorded on video tape. The experimenter was unaware of the genotype and diet group of the animals. Using Observer 4.0 (Noldus Information Technology, Wageningen, The Netherlands), frequencies and durations of the following behaviours were scored: contact: sniffing or licking any body part of the test partner; self-grooming: forepaw licking, face washing, scratching, body grooming and genital grooming; following/chasing: moving in the direction of or pursuing the test partner, who moves away; no contact: None of these behaviours. Behaviour was assessed per individual animal. Animals were used only once.

Pavlovian Fear Conditioning and Extinction

Conditioning was conducted in a home-made chamber with transparent walls and a metal rod floor. A camera was mounted on the top of the chamber. After habituation to the chamber the animals received a conditioning session consisting of a 120 s acclimation period, three pairings (60-120 s variable interstimulus interval) of the conditioned stimulus (CS) (30 s, 80 dB, 3 kHz tone) and the unconditioned stimulus (US) (1 s, 0.6 mA scrambled foot shock), in which the US was presented during the last 2 s of the CS (home-made freezing program). After a 120 s no-stimulus consolidation period the rats were returned to their home cage. 24 hrs later, initial CS-recall and subsequent CS-extinction (test 1) was measured in a novel context (white walls and a solid-Plexiglas, opaque floor) housed in a different room from that used for conditioning. After a 120 s acclimation period, the rats received five 30 s CS presentations (60-120 s variable interstimulus interval). The same procedure was repeated 24 (test 2) and 48 (test 3) hrs later to assess extinction. Freezing (no visible movement except respiration) was scored using Observer 4.0 (Noldus Information Technology) by a trained observer who was blinded to treatment conditions. Freezing was summed up in each session, and freezing during extinction tests 2 and 3 was expressed as % of freezing during test 1.

### Immunohistochemistry

Immunostaining.

The procedure was adopted from [38, 39]. Anesthetized rats were transcardially perfused with 0.1M PBS, pH 7.3, followed by 400 ml 4% paraformaldehyde dissolved in 0.1M PB, pH 7.2. Subsequently, the brains were removed from the skull and post fixed overnight in 4% paraformaldehyde at 4° C. Before sectioning, the brains were cryoprotected with 30% sucrose in 0.1 M PB. Brain sections were cut on a freezing microtome, thickness 40 μm, and collected in 6 parallel series in 0.1 M PBS containing 0.1% sodium azide. One series of each rat was used for every staining. The free-floating sections were washed three times in PBS and pre-incubated with 0.3% perhydrol (30% H2O2, Merck, Darmstadt, Germany) for 30 min. After washing three times in PBS the sections were presoaked for 30 min in an incubation medium consisting of PBS with 0.1% bovine serum albumin and 0.5% Triton X-100. The sections were incubated with goat anti-DCX (C-18 terminal; 1:3000; Santa Cruz Biotechnology Inc, Santa Cruz, Calif., USA) overnight at RT on a shaker. The sections were incubated for 90 min at RT with donkey anti-rabbit (1:1500 in incubation medium, Jackson ImmunoResearch Laboratories, West Grove, Pa., USA) and for 90 min at RT with ABC-elite, diluted 1:800 in PBS (Vector Laboratories, Burlingame, Calif., USA). In between incubations, sections were rinsed three times with PBS. The DCX-antibody peroxidase complex was made visible using 3,3′-diaminobenzidine tetrahydrochloride (DAB) staining. Sections were incubated for 10 min in a chromogen solution consisting of 0.02% DAB and 0.03% Ni-ammonium sulfate in 0.05M Tris-buffer (pH 7.6), and subsequently for 10 min in chromogen solution containing 0.006% hydrogen peroxide. This resulted in a blue-black staining. Then the sections were rinsed three times in PBS and mounted on gelatin chrome alum-coated glass slides, dried overnight in a stove at 37° C., dehydrated in an increased series of ethanol, cleared in xylene, embedded with Entellan (Merck), and cover slipped.

Quantification.

Numbers of DCX-immunopositive cells were quantified using the software program Stereo Investigator (MicroBrightfield Inc, Williston, Vt., USA). Cells were counted in the hippocampus in sections at stereotactic coordinates Bregma −3.30 mm, −3.80 mm and −4.16 mm, at 20× magnification. The results for each subject were expressed as the total amount of cells counted in these three sections added together. Hippocampal volume was estimated using photo's of the hippocampus at stereotactic coordinate Bregma −3.30 mm taken at 2.5× magnification. Using the public domain image processing program ImageJ, we subsequently drew a contour around the hippocampus of which the surface area was calculated.

### Statistical Analyses

Data are presented as mean±standard error of the mean (S.E.M.). All statistical analyses were performed using the Statistical Package for the Social Sciences version 16.0 for Windows (SPSS Inc., Chicago, Ill., USA). Data were analyzed using two-way ANOVA or repeated measures ANOVA (fear extinction recall) with Genotype and Diet as between-subject factors. Significant Genotype x Diet interactions were further analyzed using Student's t-test. Probability values of p<0.05 were considered significant. NS=not significant.

### Results

### Mixed PUFA Diet Exhibits Anxiolytic Properties in the Elevated Plus Maze in 5-HTT^{-/-} Rats

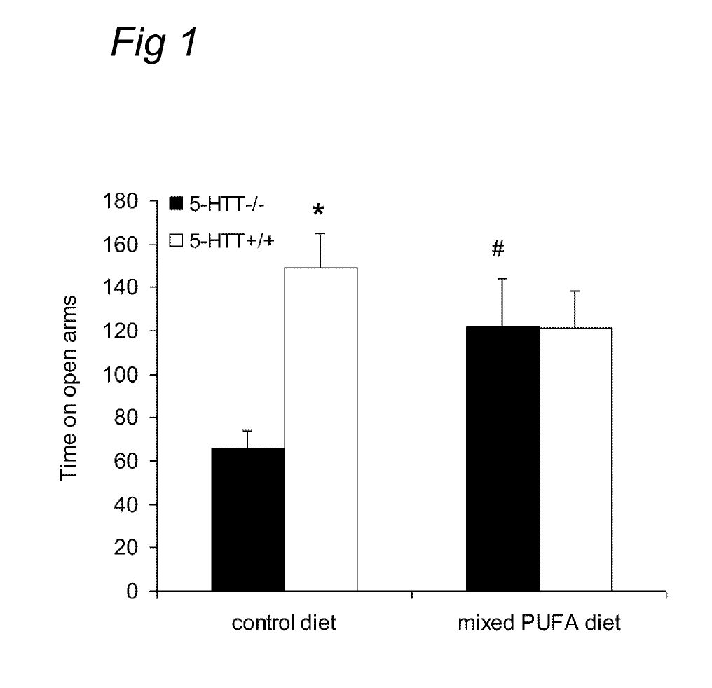

Two-way ANOVA revealed a Genotype effect (F_{(3,16)}=9.69, p<0.01) and a Genotype x Diet interaction (F_{(3,36)}=9.77, p<0.01) for time spent in the open arms, but no main effect of Diet (F_{(3,36)}=0.93, NS) was obtained (FIG. 1). 5-HTT^{-/-} rats on the control diet spent significantly less time in the open arms of the plus maze than their 5-HTT^{+/+} counterparts (t₍₈₎=4.2, p<0.005). The mixed PUFA diet increased the time spent in the open arms in the 5-HTT^{-/-} group (48)=3.2, p<0.05), while 5-HTT^{+/+} rats were unaffected (p>0.05).

### Mixed PUFA Diet Exhibits Antidepressant-Like Properties in the Forced Swim Test in 5-HTT^{-/-} Rats

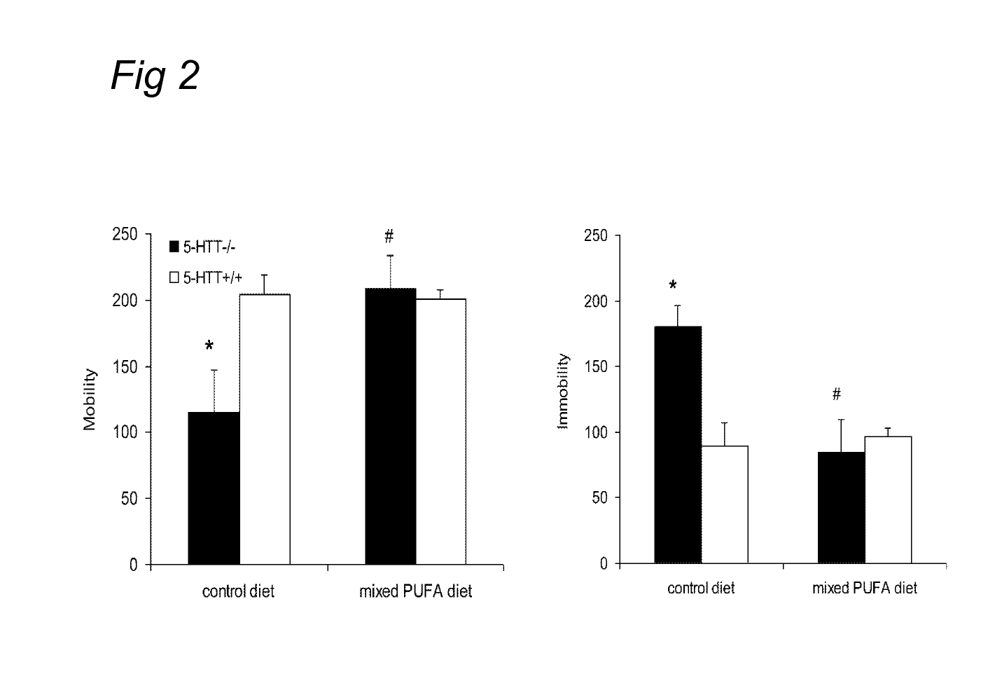

When assessing swimming behaviour (mobility) in the forced swim test, we observed significant effects of Genotype (F_{(3,12)}=5.41, p<0.05), and Diet (F_{(3,12)}=6.90, p<0.05), and a Genotype x Diet interaction (F_{(3,12)}=9.15, p<0.05) (FIG. 2a). Significantly higher mobility time were noted for 5-HTT^{+/+} rats compared to 5-HTT^{-/-} rats, both on control diet (t₍₇₎=4.7, p<0.01). Within the 5-HTT^{-/-} rats, a significant increase in mobility time was found in the mixed PUFA diet group compared to the control diet group (t₍₅₎=3.3, p<0.05); this effect of Diet was not found in the 5-HTT^{+/+} rats (p>0.05). Effect sizes for time spent floating (immobility) were overall similar. Thus, there were Genotype (F_{(3,12)}=6.54, p<0.05), Diet (F_{(3,12)}=7.90, p<0.05), and Genotype x Diet interaction effects (F_{(3,12)}=8.74, p<0.05) (FIG. 2b). These effects were due to a significant decrease in immobility time in 5-HTT^{+/+} rats compared to 5-HTT^{-/-} rats fed the control diet (t₍₇₎=4.0, p<0.01), and a significant difference between 5-HTT^{-/-} rats fed the mixed PUFA diet compared to 5-HTT^{-/-} rats fed the control diet (t₍₅₎=3.4, p<0.05), without difference between 5-HTT^{+/+} rats on control and mixed PUFA diets (p>0.1).

### Mixed PUFA Diet Enhances Social Behaviour in 5-HTT^{-/-} Rats

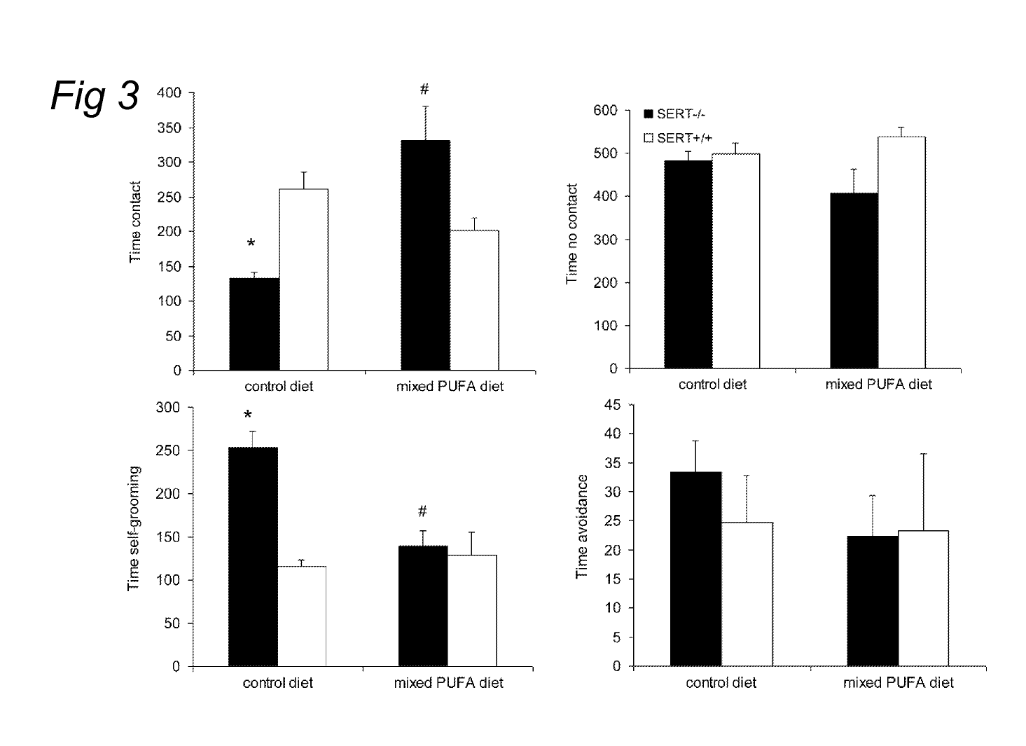

When analyzing the total time spent in contact with the test partner in the social interaction test we found a Genotype x Diet interaction (F_{(3,12)}=14.27, p<0.005) (FIG. 3A). In addition, there was a Diet effect (F_{(3,12)}=5.32, p<0.05), but no main effect of Genotype (F_{(3,12)}=0.01, NS). The 5-HTT^{+/+} rats on the control diet spent significantly more time in contact than 5-HTT^{-/-} rats on control diet during this test (t₍₆₎=4.8, p<0.005). Further, the mixed PUFA diet significantly increased contact time in the 5-HTT^{-/-} genotype (t₍₆₎=3.8, p<0.01). There was no significant difference in contact time between the control and mixed PUFA diet groups for the 5-HTT^{+/+} rats (p>0.05). None of these effects were found in the no-contact parameter (Genotype (F_{(3,12)}=4.56, NS); Diet (F_{(3,12)}=0.24, NS); Genotype x Diet interaction: (F_{(3,12)}=2.67, NS)) (FIG. 3B).

When comparing the time spent on self-grooming we found a Genotype x Diet interaction (F_{(3,12)}=10.98, p<0.01), a Diet effect (F_{(3,12)}=6.99, p<0.05), and a Genotype effect (F_{(3,12)}=14.90, p<0.005) (FIG. 3C). More time was spent on grooming by 5-HTT^{+/+} compared to 5-HTT^{-/-} when both fed on control diet (t₍₆₎=6.6, p<0.005). Comparing mixed PUFA diet fed 5-HTT^{-/-} subjects to control diet counterparts revealed that the latter group spent significantly more time on self-grooming (t₍₆₎=4.2, p<0.005). This diet effect was not seen in 5-HTT^{+/+} animals (p<0.05).

### Mixed PUFA Diet Facilitates Fear Extinction in 5-HTT^{-/-} Rats

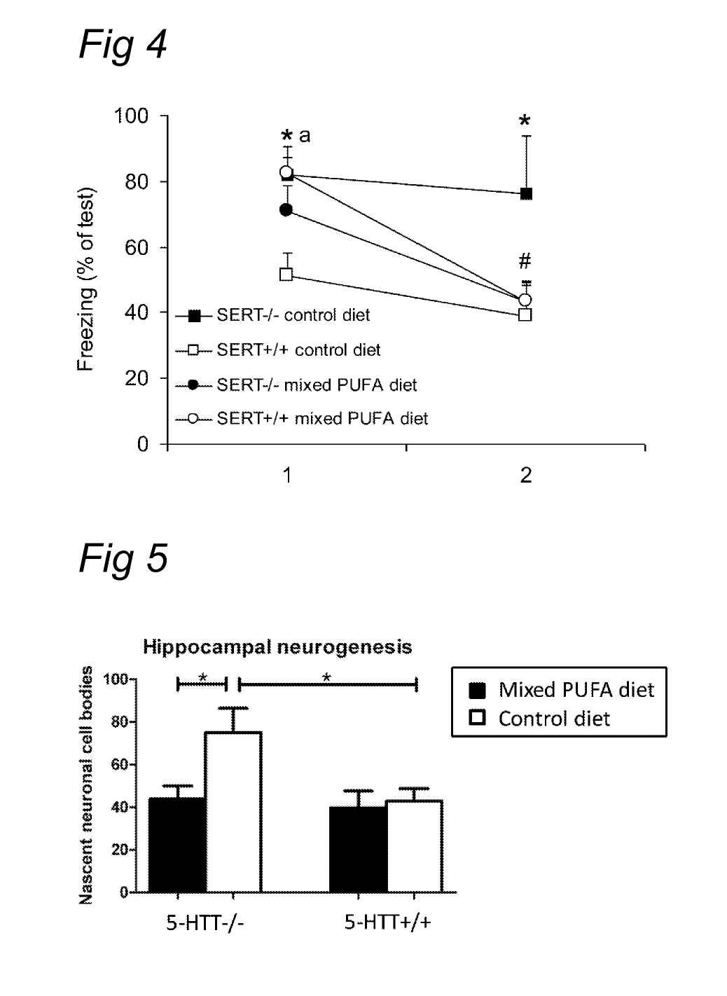

Repeated measures ANOVA for time spent freezing, expressed as % of freezing during the initial recall test (test 1) revealed a Genotype x Diet interaction (F_{(3,12)}=9.42, p<0.05), and a Genotype effect (F_{(3,12)}=5.06, p<0.05), but no Diet effect (F_{(3,11)}=0.25, NS) (FIG. 4). Thus, the mixed PUFA diet facilitated fear extinction in the 5-HTT^{-/-} rats that otherwise show impaired fear extinction. The Diet effect in 5-HTT^{+/+} rats (t₍₆₎=3.5, p<0.05) was absent during test 3 (t₍₆₎=2.3, NS), while the Genotype effect in animals on control diet during test 2 (t₍₇₎=2.7, p<0.05) was maintained throughout test 3 (t₍₇₎=2.9, p<0.05). Furthermore, the Diet effect in 5-HTT^{-/-} rats was only observed during test 3 (test 2: t₍₆₎=0.93, NS; test 3: t₍₆₎=2.4, p<0.05).

### Mixed PUFA Diet Normalizes Hippocampal Neurogenesis in 5-HTT^{-/-} Rats

Hippocampal neurogenesis showed a Genotype x Diet interaction (F_{(3,31)}=4.51, p<0.05) (FIG. 5), as well as a main effect of Diet (F_{(3,31)}=6.42, p<0.05). No effect of Genotype was found (F_{(3,31)}=3.93, NS). 5-HTT^{-/-} animals exhibited more DCX immunostaining than 5-HTT^{+/+} rats, when both were fed the control diet (t₍₁₅₎=2.42, p<0.05). The comparison of Diet effects in 5-HTT^{-/-} animals yielded a significant reduction in DCX-immunopositive hippocampal neurons in the animals fed the mixed PUFA diet (t₍₁₇₎=3.38, p<0.005). In 5-HTT^{+/+} animals, the diet had no effect on neurogenesis (t₍₁₅₎=0.32, NS).

### Mixed PUFA Diet Abolishes Differences in Hippocampal Volume Between 5-HTT^{+/+} and 5-HTT^{-/-} Rats

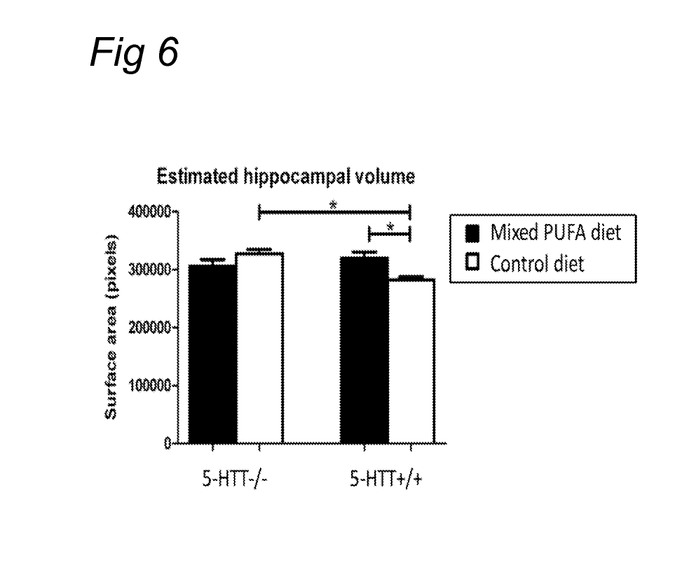

Hippocampal volume showed a Genotype x Diet interaction (F_{(3,31)}=6.52, p<0.05) (FIG. 6). No significant effects were found for Diet (F_{(3,31)}=1.42, NS) or Genotype (F_{(3,31)}=1.37, NS). Hippocampal volume was larger in 5-HTT^{-/-} rats compared to 5-HTT^{+/+} rats (t₍₁₅₎=2.98, p<0.001), and a significant enlargement of the hippocampus in 5-HTT^{+/+} rats as a result of administering the mixed PUFA diet (t₍₁₄₎=3.05, p<0.001). Animals from both genotypes on the mixed PUFA diet did not differ in hippocampal volume (t₍₁₆₎=0.84, NS).

### DISCUSSION

We have investigated the effects of a mixed PUFA diet in animals known to display elevated levels of anxiety, as well as depressive-like symptoms. The 5-HTT^{-/-} rats in the present study displayed increased levels of anxiety as evidenced by the reduced time spent on the open arms of the elevated plus maze and by a slower extinction of conditioned fear. Similarly, the 5-HTT^{-/-} rats in the present study displayed depressive-like behaviour as evidenced by increased immobility in the forced swim test [38] and reduced social exploration [40, 41]. In line with previous observations in another animal model of depression [32], related to Alzheimer's disease, the depressive-like symptoms in the 5-HTT^{-/-} rats were reduced by the mixed PUFA diet, comprising ω-3-PUFAs, phospholipids, and B-vitamins. In addition, we now show for the first time that the same dietary intervention completely abolished the anxiety in these animals. The diet-induced changes in behaviour were accompanied by a normalization of hippocampal neurogenesis in 5-HTT^{-/-} rats, and an increase in hippocampal volume in wild type rats. The present data suggest that the combined administration of ω-3-PUFAs, phospholipids, and B-vitamins may be used to treat both anxiety and depression and may help to normalize abnormalities in brain neurogenesis.

### Behavioural Tests

As expected and previously demonstrated, 5-HTT^{-/-} animals spent significantly less time on the open arms of the elevated plus maze, and failed to extinguish a fear conditioned response [42]. These behavioural manifestations correspond to increased anxiety, behavioural despair, reduced sociability and PTSD (post-traumatic stress disorder)-like emotionality respectively, and are well-documented features of major depressive disorder as well as other affective disorders [43-48]. Interestingly, while 5-HTT^{-/-} rats do not respond to SSRIs [41], the mixed PUFA diet alleviated these symptoms to such a degree that 5-HTT^{-/-} and 5-HTT^{+/+} behaviour was indistinguishable. These data suggest that mixed PUFA diets can serve as an alternative to treat anxiety and depression-like symptoms in SSRI non-responsive subjects, in particular those characterized by inherited reduced 5-HTT function.

Of special interest is the finding that the mixed PUFA diet significantly increased total time spent in contact with an unfamiliar partner during the social interaction test in 5-HTT^{-/-} animals. The reduced social interaction of 5-HTT^{-/-} rats has been linked to an increased risk of developing autism [48, 49]. In this light, our findings are in line with previously found beneficial effects of ω-3-PUFA treatment in children suffering from autism [50]. Although it remains to be determined whether individual differences in responsiveness to this treatment were linked to 5-HTTLPR genotype, the pathways that contribute to improved social behaviour as a result of prolonged ω-3-PUFA administration may be similar.

### Neurogenesis

In analogy with the behavioural data, the mixed PUFA diet effectively adjusted hippocampal neurogenesis in 5-HTT^{-/-} rats, bringing the amount of DCX immunoreactive neurons back to the level found in 5-HTT^{+/+} rats. While results are very similar, DCX holds some key advantages over the ‘gold standard’ bromodeoxyuridine (BrdU): it does neither require staining for a secondary marker in order to identify neurons, nor the injection of a mutagenic substance in living animals. DCX has been extensively validated as a marker of neurogenesis [35, 51].

The observation that DCX immunoreactivity was increased in 5-HTT^{-/-} rats and reduced by the mixed PUFA diet in these animals is paradoxical with reports that worsening of depressive symptoms is paired with a decline in hippocampal volume, and that successful treatment with SSRI's is associated with a 5HT_{1A}-dependent increase in hippocampal neurogenesis [14]. We have postulated a number of hypotheses regarding this dissociation between hippocampal neurogenesis and decreased depressive symptoms:

1] Although 5HT_{1A} receptor-affinity may be decreased in 5-HTT^{-/-} rodents [33, 41], it is possible that 5HT_{1A} signaling is still higher in 5-HTT deficient rodents compared to 5-HTT^{+/+} animals, due to increased extracellular 5-HT levels [33, 41]. Since increased 5HT_{1A} signaling may contribute to hippocampal neurogenesis, this may explain the augmented neurogenesis seen in the 5-HTT^{-/-} rats. The reversal of this phenomenon as a result of the mixed PUFA diet is in line with reported increases in 5HT_{1A} receptor binding following multi-nutrient dietary intervention [21]. Presumably, there is an optimum level of 5-HT_{1A} signaling for the regulation of hippocampal neurogenesis and the mixed PUFA diet may help to restore this.
2] The effects of ω-3-PUFA on neurogenesis in the 5-HTT^{-/-} rat may also be explained by vascular changes. Namely, 5-HTTLPR s-allele is a risk factor for vascular diseases, and s-allele depressed patients are characterized by vascular abnormalities [53]. Ischemic conditions can—under certain conditions—increase the level of neurogenesis, as seen in recovering stroke patients. ω-3-PUFA intake may have positive effects on vascular parameters like cerebral perfusion, thereby possibly reducing the level of ischemia-induced hippocampal neurogenesis. Improved vascular conditions could improve the survival rate of newly born neurons, allowing them to integrate in existing neuronal systems and contribute to the improvement of mood and cognition. Assessing the effect of dietary treatment on cerebral perfusion and survival and integration of newly developed neurons in 5-HTT^{-/-} rats could shed more light on any possible relation between these parameters.
3] 5-HTT^{-/-} rats show a reduction in the amount of brain-derived neurotrophic factor (BDNF) in the hippocampus and prefrontal cortex [54]. This implies that that survival of hippocampal neurons is reduced, since the two are believed to be closely associated [55]. It is possible that the reduction in BDNF leads to impaired survival of newly formed neurons, reducing the effectiveness of the neurogenesis. The increased neural proliferation in 5-HTT^{-/-} rats as observed in the present study may reflect a compensatory mechanism. Since ω-3-PUFA supplementation is known to increase the level of hippocampal BDNF [56-58], neuronal survival may have improved, reducing the need for additional neurogenesis. As such, Wellman et al [42] proposed that impaired fear extinction in 5-HTT^{-/-} mice is caused by a disturbance in the modulatory function of the prefrontal cortex with regards to amygdala activity. This is partially compensated through elongation of dendrites originating from the infralimbic cortex, although this is not sufficient to normalize the impaired extinction of conditioned fear response in 5-HTT^{-/-} animals. Investigating the ability of these new neurons to survive and integrate in existing neural networks may prove useful in testing this hypothesis.

In conclusion, combined administration of ω-3-PUFAs, phospholipids and B-vitamins has a profound antidepressant and anxiolytic effect in 5-HTT^{-/-} rats, as well as a normalizing effect on the increased neurogenesis seen in this genotype. Although the mechanisms driving these beneficial effects require further investigation, the results of the present study clearly suggest this dietary intervention as a putative therapeutic intervention in human patients. In particular, given the resemblances between 5-HTT^{-/-} rodents and the s-allelic variant of the 5-HTTLPR [33, 59], and meta-analyses showing that s-allele carriers respond overall poorly to SSRIs [13, 60], our findings may have heuristic value for treating SSRI-resistant patients characterized by the 5-HTTLPR s-allele.

### REFERENCES

1. Gordon, J. A. and R. Hen, The serotonergic system and anxiety. Neuromolecular Med, 2004. 5(1): p. 27-40.
2. Lesch, K. P., Genetic alterations of the murine serotonergic gene pathway: the neurodevelopmental basis of anxiety. Handb Exp Pharmacol, 2005(169): p. 71-112.
3. Mann, J. J., Role of the serotonergic system in the pathogenesis of major depression and suicidal behaviour. Neuropsychopharmacology, 1999. 21(2 Suppl): p. 99S-105S.
4. Kriegebaum, C., et al., [Serotonin now: Part 1. Neurobiology and developmental genetics]. Fortschr Neurol Psychiatr, 2010. 78(6): p. 319-31.
5. Lesch, K. P., et al., Association of anxiety-related traits with a polymorphism in the serotonin transporter gene regulatory region. Science, 1996. 274(5292): p. 1527-31.
6. Caspi, A., et al., Influence of life stress on depression: moderation by a polymorphism in the 5-HTT gene. Science, 2003. 301(5631): p. 386-9.
7. Caspi, A., et al., Genetic sensitivity to the environment: the case of the serotonin transporter gene and its implications for studying complex diseases and traits. Am J Psychiatry, 2010. 167(5): p. 509-27.
8. Canli, T. and K. P. Lesch, Long story short: the serotonin transporter in emotion regulation and social cognition. Nat Neurosci, 2007. 10(9): p. 1103-9.
9. Canli, T., et al., Neural correlates of epigenesis. Proc Natl Acad Sci USA, 2006. 103 (43): p. 16033-8.
10. Young, K. A., et al., Major depression, 5HTTLPR genotype, suicide and antidepressant influences on thalamic volume. Br J Psychiatry, 2008. 192(4): p. 285-9.
11. Frodl, T., et al., Reduced gray matter brain volumes are associated with variants of the serotonin transporter gene in major depression. Mol Psychiatry, 2008. 13(12): p. 1093-101.
12. Taylor, W. D., et al., Influence of serotonin transporter promoter region polymorphisms on hippocampal volumes in late-life depression. Arch Gen Psychiatry, 2005. 62(5): p. 537-44.
13. Serretti, A., et al., Meta-analysis of serotonin transporter gene promoter polymorphism (5-HTTLPR) association with selective serotonin reuptake inhibitor efficacy in depressed patients. Mol Psychiatry, 2007. 12(3): p. 247-57.
14. Santarelli, L., et al., Requirement of hippocampal neurogenesis for the behavioural effects of antidepressants. Science, 2003. 301(5634): p. 805-9.
15. Wang, S. H., et al., Involvement of serotonin neurotransmission in hippocampal neurogenesis and behavioural responses in a rat model of post-stroke depression. Pharmacol Biochem Behav, 2010. 95(1): p. 129-37.
16. van Praag, H., et al., Functional neurogenesis in the adult hippocampus. Nature, 2002. 415(6875): p. 1030-4.
17. Lagace, D. C., et al., Adult hippocampal neurogenesis is functionally important for stress-induced social avoidance. Proc Natl Acad Sci USA, 2010. 107(9): p. 4436-41.
18. Chalon, S., Omega-3 fatty acids and monoamine neurotransmission. Prostaglandins Leukot Essent Fatty Acids, 2006. 75(4-5): p. 259-69.
19. Suzuki, H., et al., Effect of the long-term feeding of dietary lipids on the learning ability, fatty acid composition of brain stem phospholipids and synaptic membrane fluidity in adult mice: a comparison of sardine oil diet with palm oil diet. Mech Ageing Dev, 1998. 101(1-2): p. 119-28.
20. Zimmer, L., et al., Chronic n-3 polyunsaturated fatty acid deficiency alters dopamine vesicle density in the rat frontal cortex. Neurosci Lett, 2000. 284(1-2): p. 25-8.
21. Farkas, E., et al., Dietary long chain PUFAs differentially affect hippocampal muscarinic 1 and serotonergic 1A receptors in experimental cerebral hypoperfusion. Brain Res, 2002. 954(1): p. 32-41.
22. Levant, B., et al., Decreased brain docosahexaenoic acid content produces neurobiological effects associated with depression: Interactions with reproductive status in female rats. Psychoneuroendocrinology, 2008. 33(9): p. 1279-92.
23. Beltz, B. S., et al., Omega-3 fatty acids upregulate adult neurogenesis. Neurosci Lett, 2007. 415(2): p. 154-8.
24. Cao, D., et al., Docosahexaenoic acid promotes hippocampal neuronal development and synaptic function. J Neurochem, 2009. 111(2): p. 510-21.
25. Dyall, S. C., G. J. Michael, and A. T. Michael-Titus, Omega-3 fatty acids reverse age-related decreases in nuclear receptors and increase neurogenesis in old rats. J Neurosci Res, 2010. 88(10): p. 2091-102.
26. He, C., et al., Improved spatial learning performance of fat-1 mice is associated with enhanced neurogenesis and neuritogenesis by docosahexaenoic acid. Proc Natl Acad Sci USA, 2009. 106(27): p. 11370-5.
27. Kawakita, E., M. Hashimoto, and O. Shido, Docosahexaenoic acid promotes neurogenesis in vitro and in vivo. Neuroscience, 2006. 139(3): p. 991-7.
28. Hibbeln, J. R., et al., Healthy intakes of n-3 and n-6 fatty acids: estimations considering worldwide diversity. Am J Clin Nutr, 2006. 83(6 Suppl): p. 1483S-1493S.
29. Lin, P. Y., S. Y. Huang, and K. P. Su, A Meta-Analytic Review of Polyunsaturated Fatty Acid Compositions in Patients with Depression. Biol Psychiatry, 2010.
30. Skarupski, K. A., et al., Longitudinal association of vitamin B-6, folate, and vitamin B-12 with depressive symptoms among older adults over time. Am J Clin Nutr, 2010. 92(2): p. 330-5.
31. Nemets, B., Z. Stahl, and R. H. Belmaker, Addition of omega-3 fatty acid to maintenance medication treatment for recurrent unipolar depressive disorder. Am J Psychiatry, 2002. 159(3): p. 477-9.
32. Broersen, L. M., et al., Multi-nutrient dietary intervention but not omega-3 PUFA-rich fish oil by itself reduces depression relevant to Alzheimer's disease, in ICAD. 2008.
33. Kalueff, A. V., et al., Conserved role for the serotonin transporter gene in rat and mouse neurobehavioral endophenotypes. Neurosci Biobehav Rev, 2010. 34(3): p. 373-86.
34. Kuehner, C., Gender differences in unipolar depression: an update of epidemiological findings and possible explanations. Acta Psychiatr Scand, 2003. 108(3): p. 163-74.
35. Brown, J. P., et al., Transient expression of doublecortin during adult neurogenesis. J Comp Neurol, 2003. 467(1): p. 1-10.
36. Smits, B. M., et al., Generation of gene knockouts and mutant models in the laboratory rat by ENU-driven target-selected mutagenesis. Pharmacogenet Genomics, 2006. 16(3): p. 159-69.
37. Reeves, P. G., F. H. Nielsen, and G. C. Fahey, Jr., AIN-93 purified diets for laboratory rodents: final report of the American Institute of Nutrition ad hoc writing committee on the reformulation of the AIN-76A rodent diet. J Nutr, 1993. 123(11): p. 1939-51.
38. Olivier, J. D., et al., A study in male and female 5-HT transporter knockout rats: an animal model for anxiety and depression disorders. Neuroscience, 2008. 152(3): p. 573-84.
39. Nonkes, L. J., et al., Orbitofrontal cortex and amygdalar over-activity is associated with an inability to use the value of expected outcomes to guide behaviour in serotonin transporter knockout rats. Neurobiol Learn Mem, 2010. 94(1): p. 65-72.
40. Homberg, J. R., et al., Serotonin transporter deficiency in rats improves inhibitory control but not behavioural flexibility. Eur J Neurosci, 2007. 26(7): p. 2066-73.
41. Homberg, J. R., et al., Acute and constitutive increases in central serotonin levels reduce social play behaviour in peri-adolescent rats. Psychopharmacology (Berl), 2007. 195(2): p. 175-82.
42. Wellman, C. L., et al., Impaired stress-coping and fear extinction and abnormal corticolimbic morphology in serotonin transporter knock-out mice. J Neurosci, 2007. 27(3): p. 684-91.
43. Cryan, J. F., A. Markou, and I. Lucki, Assessing antidepressant activity in rodents: recent developments and future needs. Trends Pharmacol Sci, 2002. 23(5): p. 238-45.
44. Pellow, S. and S. E. File, Anxiolytic and anxiogenic drug effects on exploratory activity in an elevated plus-maze: a novel test of anxiety in the rat. Pharmacol Biochem Behav, 1986. 24(3): p. 525-9.
45. File, S. E., et al., Animal tests of anxiety. Curr Protoc Neurosci, 2004. Chapter 8: p. Unit 83.
46. Milad, M. R., et al., Fear extinction in rats: implications for human brain imaging and anxiety disorders. Biol Psychol, 2006. 73(1): p. 61-71.
47. Rodgers, R. J. and A. Dalvi, Anxiety, defence and the elevated plus-maze. Neurosci Biobehav Rev, 1997. 21(6): p. 801-10.
48. Moy, S. S., et al., Social approach in genetically engineered mouse lines relevant to autism. Genes Brain Behav, 2009. 8(2): p. 129-42.
49. Huang, C. H. and S. L. Santangelo, Autism and serotonin transporter gene polymorphisms: a systematic review and meta-analysis. Am J Med Genet B Neuropsychiatr Genet, 2008. 147B(6): p. 903-13.
50. Meiri, G., Y. Bichovsky, and R. H. Belmaker, Omega 3 fatty acid treatment in autism. J Child Adolesc Psychopharmacol, 2009. 19(4): p. 449-51.
51. Rao, M. S. and A. K. Shetty, Efficacy of doublecortin as a marker to analyse the absolute number and dendritic growth of newly generated neurons in the adult dentate gyrus. Eur J Neurosci, 2004. 19(2): p. 234-46.
52. Schmitt, A., et al., Adult neurogenesis in serotonin transporter deficient mice. J Neural Transm, 2007. 114(9): p. 1107-19.
53. Ramasubbu, R., Serotonin transporter gene functional polymorphism: a plausible candidate gene for increased vascular risk in depression. Med Hypotheses, 2003. 61(1): p. 36-44.
54. Molteni, R., et al., Reduced function of the serotonin transporter is associated with decreased expression of BDNF in rodents as well as in humans. Neurobiol Dis, 2010. 37(3): p. 747-55.
55. Choi, S. H., et al., Regulation of hippocampal progenitor cell survival, proliferation and dendritic development by BDNF. Mol Neurodegener, 2009. 4: p. 52.
56. Venna, V. R., et al., PUFA induce antidepressant-like effects in parallel to structural and molecular changes in the hippocampus. Psychoneuroendocrinology, 2009. 34(2): p. 199-211.
57. Jiang, L. H., et al., The influence of orally administered docosahexaenoic acid on cognitive ability in aged mice. J Nutr Biochem, 2009. 20(9): p. 735-41.
58. Cysneiros, R. M., et al., Qualitative analysis of hippocampal plastic changes in rats with epilepsy supplemented with oral omega-3 fatty acids. Epilepsy Behav, 2010. 17(1): p. 33-8.
59. Hariri, A. R. and A. Holmes, Genetics of emotional regulation: the role of the serotonin transporter in neural function. Trends Cogn Sci, 2006. 10(4): p. 182-91.
60. Lesch, K. P. and L. Gutknecht, Pharmacogenetics of the serotonin transporter. Prog Neuropsychopharmacol Biol Psychiatry, 2005. 29(6): p. 1062-73.
61. Hooijmans, C. R., et al., DHA and cholesterol containing diets influence Alzheimer-like pathology, cognition and cerebral vasculature in APPswe/PS1dE9 mice. Neurobiol Dis, 2009. 33(3): p. 482-98.
62. Freund-Levi, Y., et al., Omega-3 fatty acid treatment in 174 patients with mild to moderate Alzheimer disease: OmegAD study: a randomized double-blind trial. Arch Neurol, 2006. 63 (10): p. 1402-8.
63. Bousquet, M., et al., Beneficial effects of dietary omega-3 polyunsaturated fatty acid on toxin-induced neuronal degeneration in an animal model of Parkinson's disease. Faseb J, 2008. 22 (4): p. 1213-25.
64. Su, H. M., Mechanisms of n-3 fatty acid-mediated development and maintenance of learning memory performance. J Nutr Biochem, 2010. 21 (5): p. 364-73.

1-16. (canceled)

17. A method for the prevention or treatment of anxiety or depression in a subject, or for regulating neurogenesis in a subject, wherein said subject is non-responsive to SSRI medication, comprising administering to said subject a composition comprising:
(a) at least one ω-3 polyunsaturated fatty acid (PUFA);
(b) at least two phospholipids selected from the group consisting of phosphatidylserine, phosphatidylinositol, phosphatidylcholine and phosphatidylethanolamine; and
(c) B vitamins and/or tryptophan.

18. The method according to claim 17, wherein the ω-3 polyunsaturated fatty acid is selected from the group consisting of eicosapentaenoic acid and docosahexaenoic acid.

19. The method according to claim 17, wherein phospholipids comprise at least phosphatidylcholine and phosphatidylethanolamine.

20. The method according to claim 17, wherein the composition comprises at least vitamin B6 and folic acid.

21. The method according to claim 17, wherein the composition further comprises (d) folic acid, vitamin B12, vitamin B6, magnesium and zinc; SAMe (S-adenosyl methionine), choline, betaine or copper; citrate; huperzine A; carnitine, vitamin B1, vitamin B5 and coenzyme Q10 or functional analogues thereof; a lipophylic thiamine source such as benfothiamine, allithiamine, fursulthiamine or octothiamine; coenzym Q10; an anti-oxidant, such as vitamin C, vitamin E, lipoic acid, selenium salts and carotenoids; and extract of gingko biloba.

22. The method according to claim 17, wherein the composition is a nutritional composition.

23. The method according to claim 22, wherein the composition comprises at least one component selected from the group of fats, proteins, and carbohydrates.

24. The method according to claim 17, for the prevention or treatment of anxiety or depression in a subject that has not been diagnosed with a vascular disorder.
